(19) United States
(12) Patent Application Publication
(10) Pub. No.: US 2015/0273146 A1
(43) Pub. Date: Oct. 1, 2015

(54) Hollow Infusion Catheter with Multiple Analyte-Sensing Electrodes

(71) Applicants: William Kenneth Ward, Portland, OR (US); Robert S. Cargill, Porland, OR (US); Sheila Benware, Clackamas, OR (US); Tyler B. Milhem, Portland, OR (US)

(72) Inventors: William Kenneth Ward, Portland, OR (US); Robert S. Cargill, Porland, OR (US); Sheila Benware, Clackamas, OR (US); Tyler B. Milhem, Portland, OR (US)

(73) Assignee: Pacific Diabetes Technologies, Inc., Portland, OR (US)

(21) Appl. No.: 14/667,533

(22) Filed: Mar. 24, 2015

Related U.S. Application Data
(60) Provisional application No. 61/972,887, filed on Mar. 31, 2014.
(60) Provisional application No. 62/017,800, filed on Jun. 26, 2014.

(51) Int. Cl. A61M 5/172; A61L 31/04; A61L 31/08; A61K 38/28; A61K 38/26

(52) U.S. Cl. CPC A61M 5/172; A61K 38/28; A61K 38/26; A61L 31/088; A61L 31/084; A61L 31/047; A61L 2420/08; C12Y 101/03004

(57) ABSTRACT

A durable device is disclosed. This device allows concurrent measurement of an analyte such as glucose, and delivery of a glucose-active drug such as insulin. In order to carry out both functions, only one tubular structure is necessary. In one embodiment of the invention, wires or rings of platinum, gold, or carbon which serve as indicating electrodes are circumferentially disposed around a tubular reference electrode. In an embodiment, the reference electrode is made up of a hollow silver or silver-coated tube. The hollow characteristic is necessary in order to allow concurrent delivery of insulin or other drug through the lumen. In order to optimize sensor accuracy, there are multiple individually-addressable indicating electrodes circumferentially disposed around the shaft.

### CROSS REFERENCE TO RELATED APPLICATIONS

The present application claims priority to U.S. Provisional Patent Application No. 61/972,887 filed 31 Mar. 2014 entitled “Hollow infusion catheter with multiple analyte sensing electrodes” and U.S. Provisional Patent Application No. 62/017/800 filed 26 Jun. 2015 entitled “A hybrid sensing catheter with wire elements and planar sheet elements: A dual use device designed for persons with insulin-treated diabetes”.

### BACKGROUND OF THE INVENTION

Physiology of Measuring Glucose at site of Insulin Delivery—In order to achieve optimal glycemic control, people with type 1 diabetes currently must use two through-the-skin devices: (1) a subcutaneous insulin infusion catheter connected to an external insulin pump, and (2) a subcutaneous glucose sensor connected to an external monitoring device. It would be quite beneficial to deliver insulin and continuously measure glucose using a single through-the-skin device in order to avoid the need for two skin punctures and two devices. When more than one percutaneous device is used, the amount of insertion pain and the risk for infection is greater than what would be expected from a single device. In addition, most people with diabetes find it cumbersome and unpleasant to wear more than one through-the-skin device.

It has been known for many decades that mammalian fat cells respond to insulin by pulling glucose in from the surrounding interstitial fluid, a process known as glucose uptake. For this reason, the traditional wisdom has been that it is not possible to measure glucose accurately at or near the site of insulin delivery in subcutaneous interstitial fluid. The belief has been that any attempt to carry out such a measurement would lead to a falsely low glucose reading. For example, the instruction manuals for commercially-available subcutaneous insulin pumps state that an insulin infusion catheter placed in subcutaneous fat must be placed far away from the site of a subcutaneous continuous glucose sensor/monitor.

However, over the last several years, articles in the scientific literature have reported that the effect of subcutaneously-delivered insulin to reduce the concentration of glucose in the interstitial fluid of subcutaneous fat appears to be minimal. This literature includes papers by Lindpointner et al (ref 1, 2) and Hermanides et al (ref 3) which measure glucose by microdialysis or microperfusion. In addition, a paper has recently appeared by Rodriguez et al showing no reduction of local glucose concentration near the site of insulin delivery. This group used amperometric glucose sensors rather than microdialysis (ref 4).

A comprehensive review paper discussing this literature appeared in 2014 (ref 5). In this article, the reasons that might explain these findings are discussed. Most importantly, it appears that the power of insulin to take up glucose into fat cells is low. In contrast, the power of insulin to stimulate glucose uptake into muscle tissue is great. Muscle is by far the most important insulin-responsive peripheral tissue responsible for glucose uptake. In other words, while it is true that fat tissue responds to insulin by pulling glucose from the interstitial fluid into the cells, this effect is very small and would be expected to exert a minimal effect to reduce the local concentration of glucose at the site of insulin delivery.

Importance of Durable Sensing Elements—With regard to amperometric glucose sensors, indicating electrodes (also known as working electrodes) and reference electrodes are often constructed using techniques often used in the semiconductor and micro-fabrication industries, including metal sputtering, metal evaporation, and printing of metallized inks. The layers of sputtered or evaporated metal layers applied over polymer substrates are typically very thin, on the order of 10-300 nanometers. Although such procedures can be automated at low cost, there are many risks inherent in such designs. Thin films are subject to flaking, cracking and/or delamination of the metal layers. For this reason, embodiments of the present invention teach the use of solid metal wires (or solid metal rings) circumferentially disposed around a central tubular structure in order to achieve electrode durability and avoid these problems.

The importance of sensor redundancy and addressability of individual sensors—In a variety of disciplines, the concept of measurement redundancy is used to increase accuracy. For example, it has been reported that an array of four glucose sensing units in close proximity led to accuracy benefits (ref 6) and that the use of 4 separate glucose sensors worn concurrently markedly reduced the frequency of very large (“egregious”) errors (ref 7).

For all of these reasons, the use of sensing redundancy is likely to be valuable for catheters designed for concurrent glucose sensing and insulin delivery. In an embodiment of the current invention, the use of multiple sensing units that are individually addressable helps to minimize “proximity error”. The proximity error refers to the situation in which the distance between sensing units and the site of insulin delivery affects sensing accuracy. To the extent that inaccuracy due to the proximity effect does exist, an algorithm that uses data from multiple sites would be expected to improve accuracy.

The topic of sensing error is especially relevant to the field of the artificial endocrine pancreas. The reduction of very egregious errors reduces the chance of serious adverse clinical outcomes, such as those due to overdelivery of insulin. In addition, multiple distributed sensing units that provide “tissue averaging” will likely provide increased accuracy in vivo. The general idea behind tissue averaging is that there is heterogeneity in tissue characteristics such as capillary density—the use of multiple sensing electrodes, distributed spatially in tissue, would be expected to average out these effects and provide a better metric of whole body glucose.

Another benefit of using multiple individually-addressable sensing units is that if there is an obviously erroneous sensing unit (e.g. from telemetry failure, wire breakage, short circuit, etc), then an algorithm can immediately remove such erroneous data but allow reliable data streams to remain included. In contrast, if all sensing elements are commoned together, any of the failures mentioned above will affect the entire sensing unit.

### SUMMARY OF THE INVENTION AND COMPARISON WITH PRIOR ART

Disclosed here is a durable device that allows concurrent measurement of an analyte such as glucose, and delivery of a glucose-active drug such as insulin. In order to carry out both functions, only one tubular structure is necessary. In one embodiment of the invention, wires or rings of platinum, gold, or carbon which serve as indicating electrodes are circumferentially disposed around a tubular reference electrode. In an embodiment, the reference electrode is made up of a hollow silver or silver-coated tube. The hollow characteristic is necessary in order to allow concurrent delivery of insulin or other drug through the lumen. In order to optimize accuracy, there are multiple individually-addressable indicating electrodes, each of which is circumferentially disposed around the shaft of the device. In an embodiment of the invention, a rigid shaft itself serves as the single, common reference electrode for the sensing catheter. The advantage of disposing the indicating electrode around a rigid structure is that the rigidity assists in penetrating the skin and minimizes the damaging effect of bending. The advantage of using a single common reference electrode is simplicity; the use of a separate reference electrode for each indicating electrode is needlessly complex and not necessary.

### BRIEF DESCRIPTION OF THE DRAWINGS

The present invention is illustrated by way of example, and not by way of limitation, in the figures of the accompanying drawings.

FIG. 1 is a side view of an embodiment of the sensing catheter invention, in which uninsulated wire coils serve as indicating electrodes and the exposed region of a silver tube or silver-coated tube serves as the reference electrode. A long coil of insulated wire encloses the exit wires that conduct the electrical sensor signal to an external electronic unit.

FIG. 2 is side view of an embodiment of the sensing catheter invention, in which uninsulated metal bands serve as indicating electrodes and the exposed region of a silver tube or silver-coated tube serves as the reference electrode. Many insulated metal bands enclose the exit wires that conduct the sensor signal to an external electronic unit.

FIG. 3 is a top view of an embodiment of the invention, a planar sheet that contains metallized regions intended to (a) contact indicating electrodes, (b) serve as interconnect (“exit”) traces leading to the proximal contact pads, and (c) proximal contact pads for the purpose of connecting to an external electronic unit. The purpose of this embodiment is to avoid bulky exit wires.

FIG. 4 is a top view of the sheet depicted in FIG. 3, after insulating material has been placed over the interconnect exit traces.

FIG. 5 is a side view of the sheet depicted in FIG. 4 after it has been wrapped into the shape of a hollow tube or wrapped around a solid tube.

FIG. 6 is a side view of an embodiment of the invention in which uninsulated wire coils, which serve as indicating electrodes, have been wrapped around, and contract, the corresponding metallized pads located in the distal end of the device.

FIG. 7 is a side view of an embodiment of the sensing catheter invention in which uninsulated metal bands, which serve as indicating electrodes, have been wrapped around each the corresponding metallized contact pads located in the distal end of the device.

FIG. 8 is top view of the electronic unit to which the sensing catheter and the drug infusion line attach. The electronic unit keeps the sensor polarized as well as capturing and storing sensor signals. In an embodiment, it is also capable of transmitting the sensor signals to a remote receiver. This unit also encloses the housing that surrounds and connects to the proximal end of the sensor.

FIG. 9 is a side view of the same unit which shows the electronic unit, the sensing catheter, the internal fluid path, and the external drug infusion line.

### DETAILED DESCRIPTION

Described here is an apparatus for creating a highly durable glucose-sensing device that also serves as a delivery conduit for a glucose-active drug. In the following description, for the purposes of explanation, numerous specific examples are provided in order to give a thorough understanding of the invention. It will be apparent, however, to one skilled in the art, that the present invention may be practiced without these specific details.

In one embodiment, the starting material for the sensing catheter is a hollow tube (for example, a tube made of stainless steel) that has an outer diameter of between 0.2-1.0 mm. If the tube is made of stainless steel, it can be used as a reference electrode without deposition of silver (Ag). However, more preferably, Ag is deposited on the surface of the tube by use of electroplating, electroless deposition, swaging, or the creation of a drawn filled tube. Alternatively, the starting material for the tube can be silver or (in order to impart more hardness than soft elemental silver) an alloy of silver. For increased flexibility and comfort, the tube can be made of a polymer on which Ag is deposited. If the central tube is made from or is covered by Ag, a layer of AgCl is typically formed on this surface by exposing the silver to ferric chloride, to an electrolysis solution containing chloride (typically HCl, KCl or both), or by applying AgCl ink.

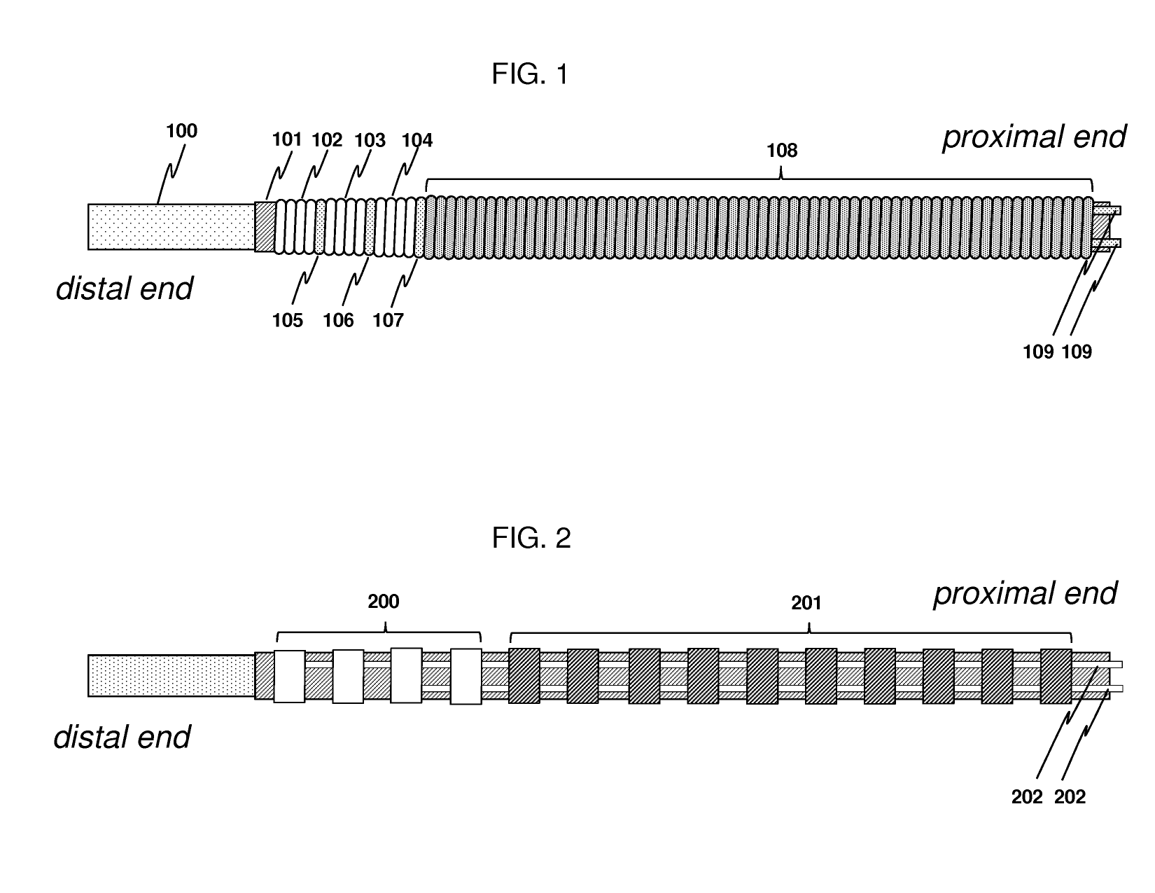

FIG. 1 is a side view of an embodiment of the sensing catheter invention in which the uninsulated region of a silver tube or silver-coated tube 100 serves as the reference electrode. The metal tube is partially covered by an insulating layer 101, which can be a polyimide, polyester, a fluoropolymer, polyurethane or other non-conductive material. To serve as indicating electrodes, at least two separate coils of platinum, gold, or carbon (or platinum-coated, gold-coated, carbon-coated) wire are placed circumferentially around the insulation 101. In the embodiment shown in this figure, there is a distal indicating electrode 102, a middle indicating electrode 103, and a proximal indicating electrode 104. In one embodiment (not shown), spaces are left between the indicating electrode coils to avoid short circuits. Alternatively, to form a mechanically stable structure, one or more wire turns of each coil can be left insulated to electrically separate the indicating electrodes. In this embodiment, there is a distal insulated wire turn 105, a middle insulated wire turn 106, and a proximal insulated wire turn 107.

Each indicating electrode coil is individually-addressable and is used ultimately to measure glucose or other analyte such as oxygen, lactic acid or other compound. As each coil is disposed on the silver-coated tube, it covers (and holds in place) the exit wire from the more proximal coil. These coiled wires are durable and flex tolerant, as wires configured in the shape of a coil tend to resist breakage due to their inherent flexibility.

More toward the proximal end of the structure, an insulated wire 108 can be coiled around the central structure for the purpose of immobilizing the exit wires 109. In this example, 3 indicating electrode coils are shown, but there can be many more or as few as two. The benefit of more than one indicating electrode coil is that (a) more than one signal can be averaged together or (b) a signal(s) can be excluding or under-weighting by an algorithm, either of which improves sensing accuracy. Toward the proximal end of the hollow tube, the exit wires 109 (originating from the indicating electrodes) emerge from under the insulated wire segment. The third exit wire is on the back side of the tube and is not visualized in this figure. Connections are made with these exit wire conductors and the silver tube for the purpose of acquiring the electrical signals in order to measure the analyte

One method of applying the wires is an automated or manual wire coiler that pays out wire from a payout spool as the central catheter structure is rotated. Such machines are known as wire servers (such as planetary wire servers). Such machines can be designed to take off insulation as needed, eg by laser ablation, application of heat, or physical methods such as cutting with a knife or abrading with an abrasive device. Alternatively, one can start with a bare wire; at intervals, before winding on to the central structure, the insulation can be applied. It should be noted that the wires used in this invention are not necessarily round wires; for example they can be flat.

Another embodiment of a durable sensing catheter is shown in FIG. 2. In this embodiment, rather than wire coils, the indicating electrodes are made from discrete rings of Pt, Au, carbon or other suitable electrode material which are applied circumferentially and attached to flat or round exit wires. In one embodiment, there are 4 indicating electrode rings 200. Similar to the situation for coiled wires, as the more proximal rings are attached, they surround and hold down the exit wires from the more distal rings. Metal rings are well known in the device industry and are often called marker rings. For example, marker rings are used as radioopaque devices to mark the location (and allow localization by radiographic imaging) of devices such as cardiac stents. In the design shown in FIG. 2, some of the more proximal marker rings are not active indicating electrodes. Instead, they are insulated rings 201 used only to hold the exit wires 202 in place.

It is important to note that there can be problems arising from the use of standard round exit wires for the connection of the distal indicating electrodes to the electronic signal acquisition unit. Specifically, going from distal to proximal, the number of exit wires increases. For this reason, the sensing catheter profile gets larger and also becomes cone-shaped, increasing the circumference as one goes from distal to proximal, as can be seen in FIG. 1.

If there were a way to avoid the need for bulky exit wires, cost would be reduced and the sensing catheter would be less cone-shaped. One solution to this problem is to create a hybrid system, without the need for bulky exit wires. With a hybrid system, the indicating electrodes are made of solid or plated metal (such as solid wire coils or solid rings) but, instead of exit wires, the conductors to the proximal region are microfabricated thin metal traces on a planar sheet.

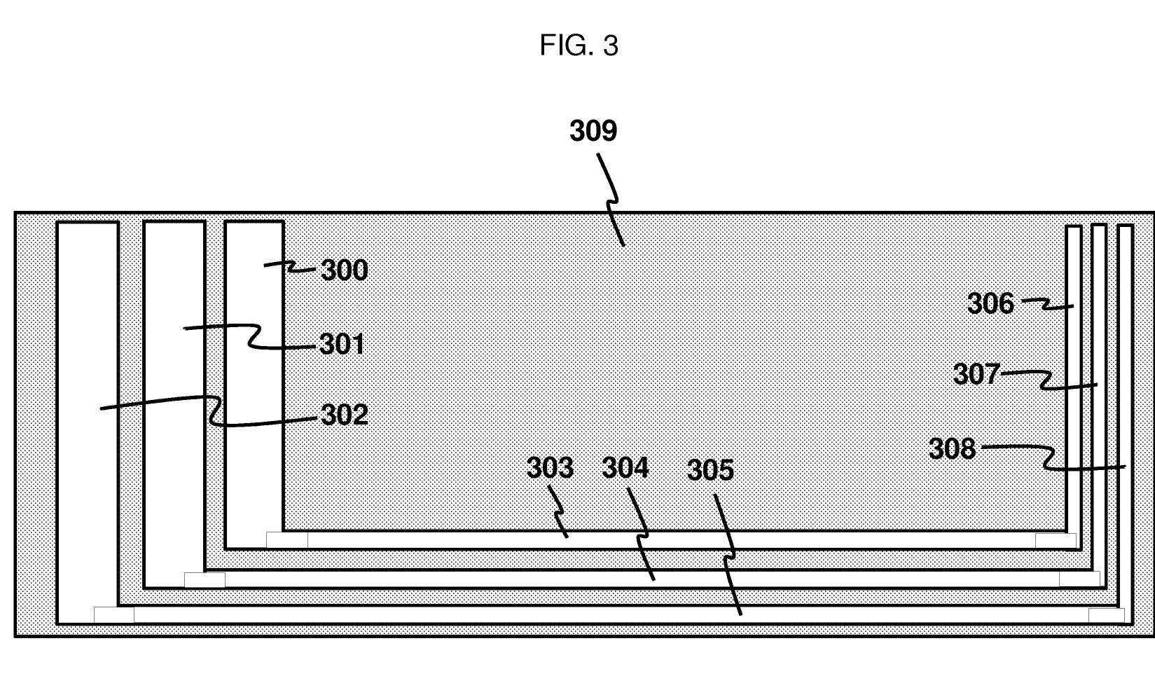

An embodiment of a planar sheet is shown in top view in FIG. 3. The sheet can be created by deposited metallized electrode contact pads 300, 301, and 302 which will be at the distal end of the catheter/needle, near where the liquid drug is released into the body. These pads are in electrical continuity with connecting exit traces 303, 304, and 305. The exit traces are typically 10-1000 nm in thickness but sometimes up to 10 microns. The exit traces are also in electrical contact with corresponding proximal contact pads 306, 307, and 308, the purpose of which is to provide contact with an external electronic unit. There are many possible choices for the composition of the metal, one of which is gold. If additional adhesion is needed, the gold layer can be placed over a nickel layer, a copper layer, or a titanium layer.

The polymer sheet 309 can be made of polyimide (e.g. thickness of 5-25 microns) or from many other polymers.

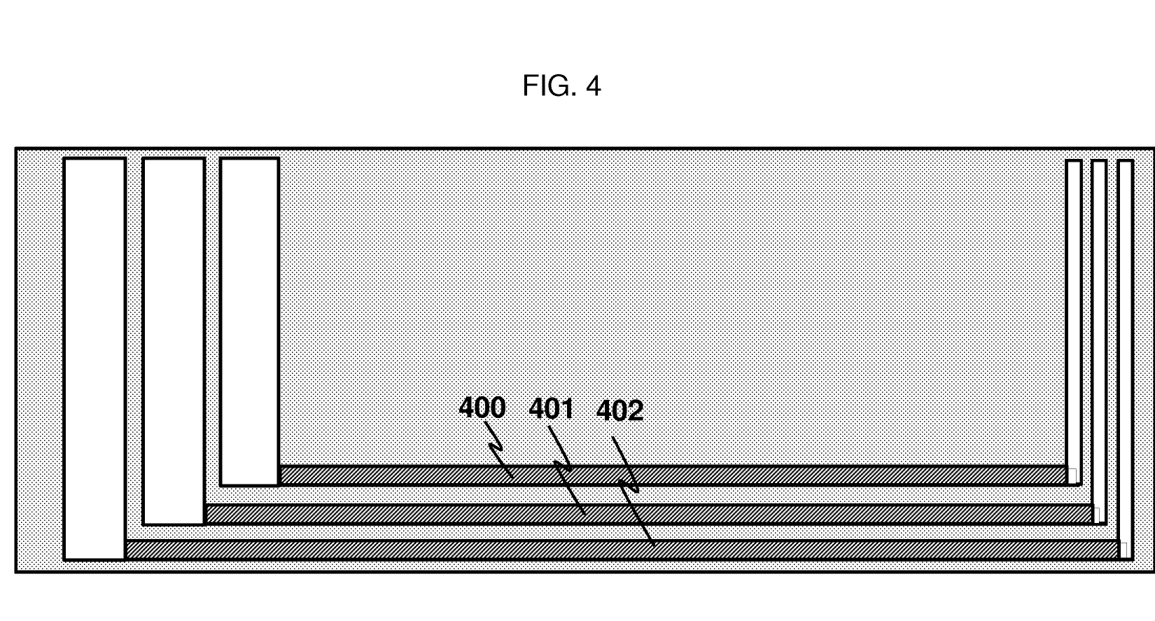

FIG. 4 shows the same sheet with insulating layers 400, 401, and 402 placed over the metal exit traces to avoid short circuits. There are many ways to create the selective insulation pattern shown in FIG. 4. In one such embodiment, one can first coat the entire planar surface with an insulating polymer, eg by spin coating or other method of coating. Many different polymers can be used for this purpose. Then, using standard photolithography techniques, the insulating polymer can be removed in patterns from the proximal and distal contact pads, in order to achieve insulation only of the exit traces. Alternatively, the insulating polymer can be applied initially through a mask to avoid polymer covering the unwanted areas. In such a case, it will not be necessary to later remove the polymer from these areas.

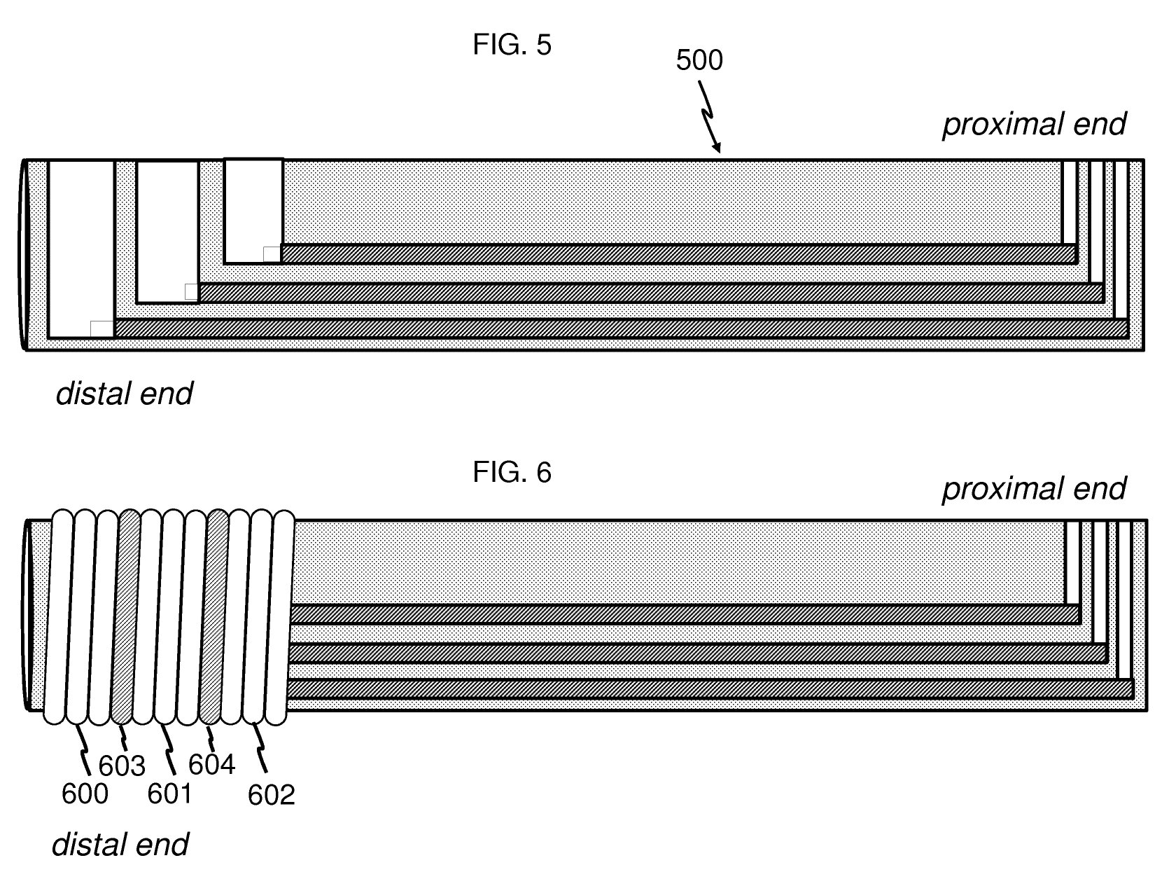

In FIG. 5, a side view, the sheet has been formed into a tube 500 or affixed around a hollow structure to form a tube 500. An adhesive can be used to affix the wrapped sheet circumferentially around a rigid tube. The hollow structure is typically 21-30 gauge (AWG) in diameter.

FIG. 6, a side view, shows this tube after robust indicating electrodes have been applied over the distal electrode contacts. In this embodiment, there is a distal uninsulated coiled wire indicating electrode (IE) 600, a middle coil IE 601, and proximal coil IE 602. In one embodiment, there are insulated wire turns 603 and 604 between the uninsulated coils to provide mechanical stability. Because the exit traces have a very low profile, the overall shape of the sensing catheter does not assume a cone shape.

There are several ways in which the indicating electrodes can then be applied. For example, platinum, gold or carbon wires (or wires coated in these elements) with a diameter of 25-100 microns can be wound as individual coils. Alternatively, the wire can be wound in one continuous coil with tight windings (e.g. 5-25 turns) making up each individual coil, with loose turns between the coils. In such a case, one can later break the connecting wires between adjacent coils (such as with the use of a laser or by mechanical means), creating the individual electrode coils. One way to avoid loosening of the connecting wires is to place a collar of heat shrinkable tubing in the region between the coils to keep the connecting wire fragments in place. Companies such as Vention, Zeus, and Cobalt Polymers sell such shrink tubing which is often made from polyolefin or polyester.

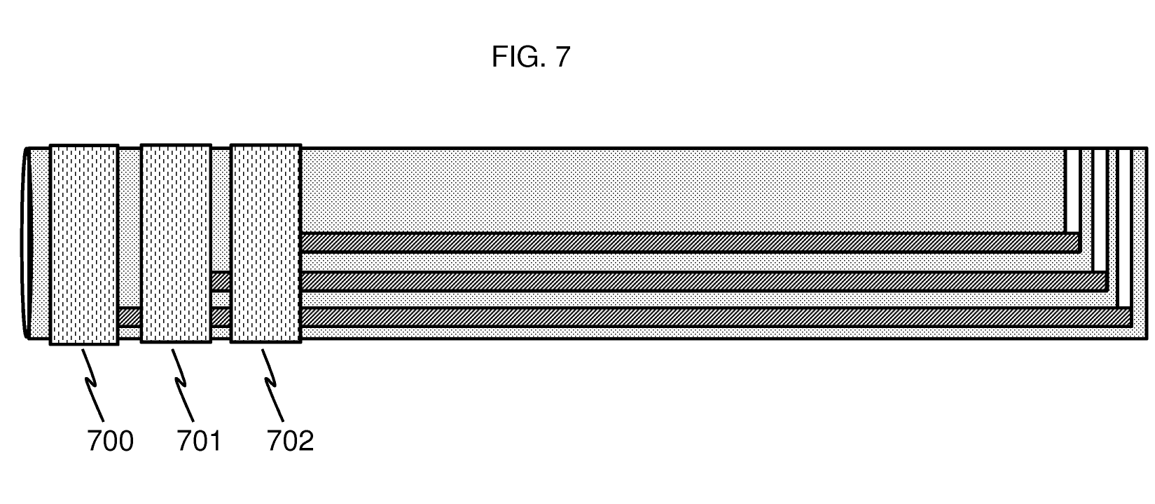

FIG. 7, a side view, shows an alternative embodiment of the sensing catheter invention. In this embodiment, instead of wires, the indicating electrodes are made from rings 700, 701 and 702. The rings can be made from Pt, Au, or carbon or can be a less expensive metal coated with Pt, Au, or carbon.

After the indicating electrodes have been placed on the tube, they can be coated with an enzyme such as glucose oxidase followed by a coat of a permselective polymer such as perflourosulfonic acid or oxygen-permeable polyurethane in order to create an electrical current signal that is based on the concentration of glucose to which the sensor is exposed. Permselective polymers such as sulfonated polyether sulfone, poly(phenylenediamine), polypyrrole, polyaniline, or polyphenol can be placed directly against the platinum indicating electrode to minimize or avoid permeation of interfering compounds such as acetaminophen, ascorbic acid or uric acid.

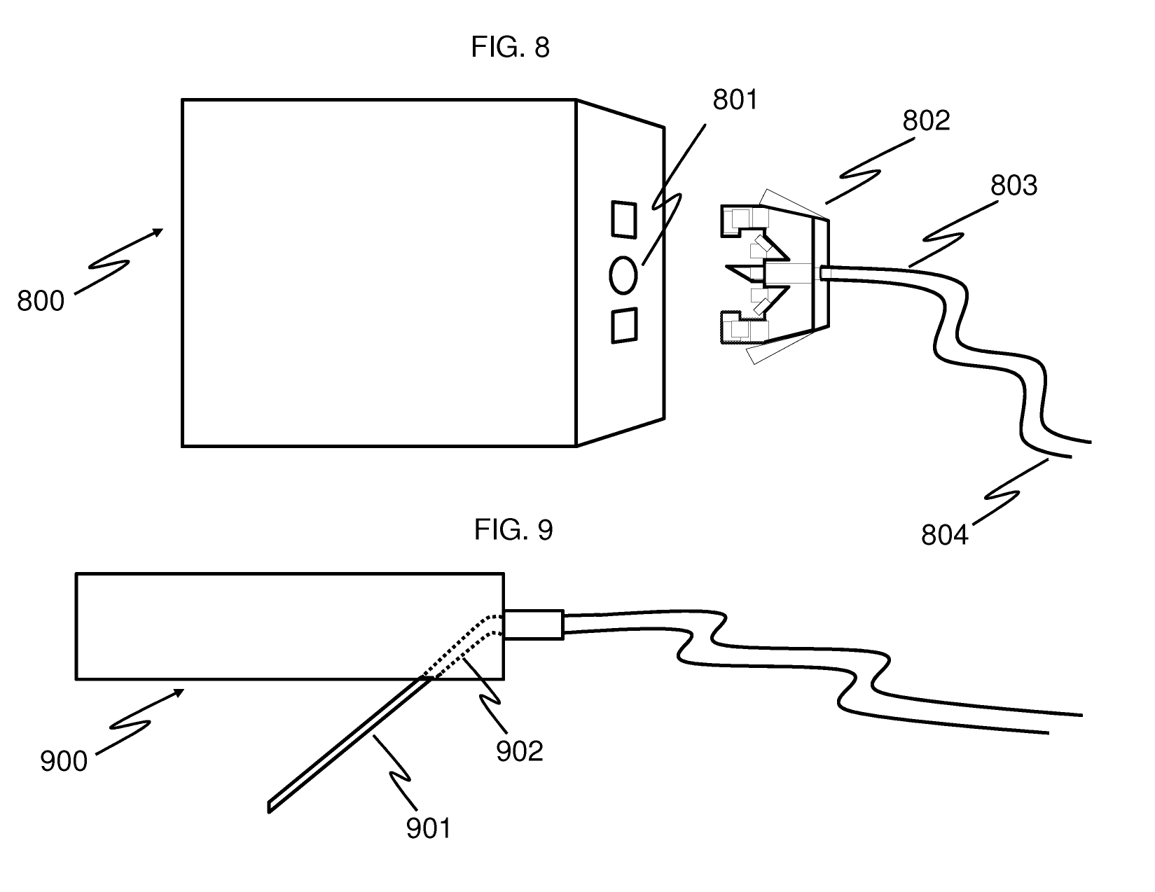

Attachment to a drug infusion device—The proximal end of the sensing catheter is configured so that it can be attached to a drug infusion line. FIG. 8 is a top view which depicts the device which, in one embodiment, is affixed to the skin. The skin-worn module 800 is a multi-purpose device that contains a drug infusion port 801 (which is in continuity with the sensing catheter, which cannot be seen in this view). A drug infusion part 802, which in one embodiment has outer stabilization posts, has a drug infusion needle which mates with the drug infusion port 801. Attached to the multi-pronged device is drug infusion tubing 803. In one embodiment, the proximal portion of the tubing 804 attaches to a drug infusion pump which is not shown.

FIG. 9 is a side view of the body-worn device 900. Shown in this view are the sensing catheter 901 which is connected to the drug infusion line through an internal hollow fluid path 902.

The drug infusion system can be used to infuse a glucose-active drug such as insulin for the purpose of controlling glucose levels in a person with diabetes mellitus. In addition, other glucose-active drugs or hormones such as glucagon or glucagon like polypeptide-1 analogs can be infused. These drugs can be used for the purpose of implementing an automated, closed loop unihormonal or bihormonal pancreas (also known as an artificial endocrine pancreas or an artificial pancreas) such as that described in Castle et al (ref 8). For use as an artificial pancreas, there is a need for glucose-controlling algorithm deployed using an electronic device which is in electronic communication with the sensing data and the pump(s) that deliver insulin or other drugs.

Compounds Applied to the Electrodes for Measurement of Glucose or other Analyte—In order to measure glucose or other analytes, several membranes or layers are typically placed over the indicating electrode coils and the silver (or silver-coated) reference electrode. For example, a specificity (or selectivity) membrane can be placed directly over the indicating electrode surface. Examples include cellulose acetate, Nafion (a perfluorosulfonic acid from Dupont), sulfonated polyether sulfone, SPEES-PES (sulfonated poly ether-ether sulfone/poly ether sulfone), or combinations of the above materials. Other examples of a specificity membranes include molecular weight cutoff membranes (the MW cutoff must be greater than 34 Daltons), or various filtration membranes that allow passage of hydrogen peroxide, including poly (1,2 phenylenediamine [PPD]), 1,3 PPD, polypyrrole, polyaniline, polyphenol or other polymer of aromatic compounds known to persons skilled in the art.

An enzyme such as glucose oxidase or glucose dehydrogenase is applied over the specificity membrane or directly against the Pt or Pt-like indicating electrode for measurement of glucose. Other enzymes (e.g. cholesterol oxidase, lactate oxidase) can be used for the measurement of other analytes. Any of these enzymes can be applied by dip coating, spray coating, electro-deposition using polarized electrodes, or other application techniques known by those skilled in the art.

Over the enzyme layer, a permselective membrane will typically be applied for the purpose of regulating entry of oxygen and glucose. Mammals have much more glucose in their subcutaneous tissue than oxygen, and since the glucose oxidase reaction requires one oxygen molecule for each glucose molecule, there is a need to favor oxygen permeation and limit glucose entry by controlling the permselectivity of the outer membrane. There are many examples of outer permselective membranes including, but not limited to, polyurethanes, silicones, acrylates, fluoropolymers and many other polymers. There may be one or more additional membranes disposed over the outer permselective membrane for the purpose of interacting with the biological mammalian environment in which the sensing catheter is placed. Such membranes can include porous membranes that enhance capillary growth in order to continue to provide oxygen and measured analytes to the sensing elements.

Typically, this sensing catheter will be placed in the subcutaneous location; however other locations may also be used such as the intravascular or intraperitoneal sites. Typically the electronics will keep the indicating electrode polarized positively with respect to the reference electrode. For the measurement of glucose or other analytes that use oxidase enzymes, the oxidation of hydrogen peroxide can be measured, such a voltage in the range of +400 to +750 mV. Alternatively, the reduction of hydrogen peroxide to water can be measured in the region of +100 mV to −200 mV. If a mediator such as Prussian blue, osmium or other mediator is used to shuttle electrons, low polarizing voltages can be used.

Distinction from other biosensor inventions—The prior art includes several other biosensor inventions that utilize circumferential electrodes. For example, the following U.S. patents teach the use of a silver reference electrode which is circumferentially disposed around a central Pt indicating electrode: U.S. Pat. No. 5,165,407 (first inventor: Wilson); U.S. Pat. No. 8,187,433 (Ward); U.S. Pat. No. 7,529,574 (Jansen); U.S. Pat. No. 7,228,162 (Ward); U.S. Pat. No. 7,146,202 (Ward); U.S. Pat. No. 8,571,625 (Kamath); U.S. Pat. No. 8,548,551 (Kamath); U.S. Pat. No. 8,483,793 (Simpson); U.S. Pat. No. 7,074,307 (Simpson); U.S. Pat. No. 8,170,803 (Kamath); and U.S. Pat. No. 7,896,809 (Simpson). Although such a design is structurally sound, one limitation of such a design is that there is only one available analyte sensing site (the central working electrode). None of these above-mentioned inventions teach (a) the robust design disclosed here in which multiple indicating electrodes are disposed around a single Ag reference electrode or (b) a design in which the sensing unit(s) is(are) integrated into a catheter or needle in order to allow concomitant fluid delivery.

In contrast, the invention of Neinast (application 20060263839, Publication Date; 2009 Nov. 23) does teach integration of sensing units into a catheter. However, this invention teaches the use of thin outer metal layers (such as would be deposited by photolithography and related techniques) that can easily become damaged; this invention does not teach the use of robust circumferential wires or rings disposed around the central tube.

U.S. Pat. No. 7,799,191 (Yu), U.S. Pat. No. 7,534,330 (Yu) and U.S. Pat. No. 8,608,922 (Papadimitra-kopoulos) teach the use of a Pt indicating electrode in the shape of a coil, but these sensing systems do not have multiple individually addressable sensing units, and does not have a hollow lumen suitable for concurrent drug delivery.

Importantly, in all embodiments of the current invention, in order to function correctly, a part of the device must reside within the body and a part must reside outside of the body. The sensing electrodes must be indwelled within the body. The electronics unit, the fluid path within the electronics unit, the drug infusion pump and the tubing for the pump must all be located on the surface of the body or otherwise outside of the body. Without the attached drug infusion device, no embodiment of the invention can function normally. Without a portion located within the body, no embodiment of the invention can function normally. Tubular structures that are designed to function without any part implanted, such as hand held blood glucose monitoring devices, cannot carry out the functions of this invention. Similarly, tubular structures that are designed to be entirely indwelled within the body cannot carry out the functions of this invention. For example, such devices cannot be connected to extracorporeal pumps.

What is claimed is:

1. A hollow elongated structure through which a glucose-active drug can be administered and which is capable of amperometrically monitoring glucose continuously,
the outer surface of which contains more than one indicating electrode,
in which the hollow structure serves as a reference electrode or a combined reference/counter electrode,
in which each indicating electrode is a wire coil consisting of solid Pt, Au, or carbon, or a coil of wire coated with Pt, Au or carbon,
in which each indicating electrode is coated with at least one enzyme and at least one polymer layer,
in which each indicating electrode is individually-addressable and not electrically connected to other indicating electrodes,
in which each indicating electrode is connected to an exit conductor that leads to the proximal region, and
which can function normally only when the distal electrode-containing part is located within a mammalian body and the proximal drug delivery part is outside of the body.

2. The invention of claim 1 in which the glucose-active drug is insulin,

3. The invention of claim 1 in which the glucose-active drug is glucagon,

4. The invention of claim 1 in which the hollow structure is made of silver or another metal coated with silver,

5. The invention of claim 1 in which the enzyme is glucose oxidase,

6. The invention of claim 1 in which the distal electrode region is implanted in the subcutaneous space, the intravascular space, or the intraperitoneal space,

7. The invention of claim 1 in which the exit conductors are round wires,

8. The invention of claim 1 in which the exit conductors are flat wires,

9. The invention of claim 1 in which the exit conductors are low profile microfabricated metal traces (less than 10 μm thickness), thus avoiding enlargement of the proximal diameter.

10. A hollow elongated structure through which a glucose-active drug can be administered and which is capable of amperometrically monitoring glucose continuously,
The outer surface of which contains more than one indicating electrode,
in which the hollow structure serves as a reference electrode or a combined reference/counter electrode,
in which each indicating electrode is a ring of solid Pt, Au, or carbon, or a ring coated with Pt, Au or carbon,
in which each indicating electrode is coated with at least one enzyme and at least one polymer layer,
in which each indicating electrode is individually-addressable and not electrically connected to other indicating electrodes,
in which each indicating electrode is connected to an exit conductor that leads to the proximal region, and
which can function normally only when the distal electrode-containing part is located within a mammalian body and the proximal drug delivery part is outside of the body.

11. The invention of claim 10 in which the glucose-active drug is insulin,

12. The invention of claim 10 in which the glucose-active drug is glucagon,

13. The invention of claim 10 in which the hollow structure is made of silver or another metal coated with silver,

14. The invention of claim 10 in which the enzyme is glucose oxidase,

15. The invention of claim 10 in which the distal electrode region is implanted in the subcutaneous space, the intravascular space, or the intraperitoneal space,

16. The invention of claim 10 in which the exit conductors are round wires,

17. The invention of claim 10 in which the exit conductors are flat wires,

18. The invention of claim 10 in which the exit conductors are low profile microfabricated metal traces (less than 10 μm thickness), thus avoiding enlargement of the proximal diameter.
